(19) United States
(12) Patent Application Publication
(10) Pub. No.: US 2014/0224834 A1
(43) Pub. Date: Aug. 14, 2014

(54) RECONSTITUTION DEVICE

(71) Applicant: GLUCAGO, LLC, El Segundo, CA (US)

(72) Inventors: Rush L. Bartlett, II, Mountain View, CA (US); Arthur L. Chlebowski, Granger, IN (US); Peter M. Greco, Hermosa Beach, CA (US)

(21) Appl. No.: 14/343,565

(22) PCT Filed: Sep. 20, 2012
(86) PCT No.: PCT/US12/56318

§ 371 Date: Mar. 7, 2014

Related U.S. Application Data
(60) Provisional application No. 61/536,865, filed on Sep. 20, 2011.
(60) Provisional application No. 61/542,415, filed on Oct. 3, 2011.
(60) Provisional application No. 61/599,140, filed on Feb. 15, 2012.

(51) Int. Cl. A61J 1/20

(52) U.S. Cl. CPC A61J 1/2093; A61J 2001/201
USPC 222/82

(57) ABSTRACT

An apparatus includes a reconstitution chamber and a diluent chamber. The reconstitution chamber is operable to hold a lyophilized material and able to combine the lyophilized material with a diluent. The diluent chamber holds a diluent and is in selective fluid communication with the reconstitution chamber.

### PRIORITY

This application claims priority to U.S. Provisional Patent Application Ser. No. 61/536,865, filed Sep. 20, 2011, entitled “DRUG VIAL THAT ALLOWS FOR RECONSTITUTION INSIDE MEDICAL INJECTION DEVICES,” U.S. Provisional Patent Application Ser. No. 61/542,415, filed Oct. 3, 2011, entitled “DRUG VIAL THAT ALLOWS FOR RECONSTITUTION INSIDE MEDICAL INJECTION DEVICES,” and U.S. Provisional Patent Application Ser. No. 61/599,140, filed Feb. 15, 2012, entitled “DRUG VIAL THAT ALLOWS FOR RECONSTITUTION INSIDE MEDICAL INJECTION DEVICES,” the disclosures of which are incorporated by reference herein.

### BACKGROUND

Therapeutic compounds often benefit from storage in a freeze dried or spray dried format in order to prolong shelf life. A solid form of a therapeutic often benefits from extended shelf life due to the removal of a portion of oxygen or water from a storage environment. Increases in the need for this type of long term storage in a solid form, through lyophilization and or spray drying, has been increasing due to the biotechnology boom in recombinant proteins, improved vaccines, and other chemical compounds which would benefit from longer term storage at higher temperatures such as military drugs which could be stockpiled. From the initial introduction of solid form storage of therapeutic compounds they are traditionally filled into a pharmaceutical vial that is cylindrical in shape and has one opening at the top and a solid glass bottom. This vial can be filled on existing fill finish equipment in the industry and then stoppered such that it maintains the shelf life of a substance in the solid form. The problem with using this filled vial is that a manual process requiring multiple steps and or secondary devices is needed to mix this dried drug with liquid diluents prior to drawing the solution up into a syringe and then administering it to the patient. Additional strategies have attempted to solve the complex and dangerous procedure of mixing and administering with this single chambered vial that has been used for the better part of the last 50 years. However, no device has succeeded in being both adaptable to existing manufacturing used for the single chamber single opening vial and also simultaneously being flexible enough to be used in a wide variety of device architectures.

Three primary strategies have generally been explored as containers for freeze or spray dried medication, including a first strategy of storing the medication in a single chambered vial with one opening. The second strategy is to store medication in a dual chamber storage system. One example of this type of dual chamber storage system is commonly known as the Pfizer Act-O-Vial system. This vial has two chambers separated by a rubber stopper and a single opening at the top. This allows for the administering person to press the top in order to dislodge the middle rubber stopper such that the liquid can mix with the lyophilized powder in the lower chamber. Then the administering person is required to draw up the mixed liquid into a syringe before administering the therapeutic compound.

A third strategy currently in use is to use a dual chambered system that has two openings. This type of system includes one opening at the top and one at the bottom of the cylindrical dual chambered storage system. This is most commonly known as a Vetter Vial. Similar to the Pfizer Act-O-Vial, the Vetter Vial has a rubber stopper bypass in the center that is activated by pressure from a device which then causes the liquid and powder to mix. This then requires the patient only to deliver the drug via the attached needle which is incorporated within the device. The Vetter Vial facilitates a much easier and useful system from a patient perspective but its critical drawback is that its dual chambered dual opening design is only compatible with custom Vetter filling machines. As a result the majority of pharmaceutical filing operations around the world are unable to use this vial system, and it is very expensive when one includes the cost of the custom filling machines.

Due to the difficulties associated with the required secondary steps for a dual chambered vial with one opening and the difficulty and high cost of filling a dual chambered vial with two openings, a common solution employed by the market is to utilize a single chambered drug vial with one opening in conjunction with an expensive or potentially dangerous secondary instrument, like a syringe or reconstitution aid, in order to administer the mixed therapeutic solution.

While a variety of reconstitution devices have been made and used, it is believed that no one prior to the inventor(s) has made or used an invention as described herein.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1 depicts a cross sectional elevation view of an exemplary reconstitution device;

FIG. 2 depicts a cross sectional elevation view of a reconstitution chamber and a diluent chamber of the reconstitution device of FIG. 1;

FIG. 3 depicts a cross sectional elevation view of the reconstitution chamber and the diluent chamber of FIG. 2 inserted into a syringe;

FIG. 4 depicts a cross sectional elevation view of the reconstitution device of FIG. 1 ready for use;

FIG. 5 depicts a cross sectional elevation view of the reconstitution device of FIG. 1 with the plunger advanced and a diluent flowing into the reconstitution chamber;

FIG. 6 depicts a cross sectional elevation view of the reconstitution device of FIG. 1 with the plunger further advanced and the diluent fully added to the reconstitution chamber;

FIG. 7 depicts a cross sectional elevation view of the reconstitution device of FIG. 1 with the plunger fully advanced and the reconstituted material urged from the syringe;

FIG. 8 depicts a cross sectional elevation view of an alternative exemplary reconstitution device;

FIG. 9 depicts a cross sectional elevation view of the reconstitution device of FIG. 8 ready for use;

FIG. 10 depicts a cross sectional elevation view of the reconstitution device of FIG. 8 with the plunger advanced and a stopper dislodged in the proximal direction;

FIG. 11 depicts a cross sectional elevation view of the reconstitution device of FIG. 8 with the plunger further advanced and the diluent fully added to the reconstitution chamber;

FIG. 12 depicts a cross sectional elevation view of the reconstitution device of FIG. 8 with the plunger fully advanced and the reconstituted material urged from the syringe;

FIG. 13 depicts a cross sectional elevation view of another alternative exemplary reconstitution device;

FIG. 14 depicts a cross sectional elevation view of a reconstitution chamber of the reconstitution device of FIG. 13;

FIG. 15 depicts a cross sectional elevation view the reconstitution device of FIG. 13 inserted into a syringe;

FIG. 16 depicts a cross sectional elevation view the reconstitution device of FIG. 13 inserted into a syringe with a rod removed;

FIG. 17 depicts a cross sectional elevation view the reconstitution device of FIG. 13 inserted into a syringe with a plunger advanced;

FIG. 18 depicts a cross sectional elevation view the reconstitution device of FIG. 13 inserted into a syringe with a plunger fully advanced;

FIG. 19 depicts a cross sectional perspective view of another alternative exemplary reconstitution device with a thread routed through the needle;

FIG. 20 depicts a cross sectional perspective view of another alternative exemplary reconstitution device with a cone shaped piercer;

FIG. 21 depicts a cross sectional perspective view of another alternative exemplary reconstitution device With a thread between a plunger and a stopper;

FIG. 22 depicts a cross sectional elevation view of another alternative exemplary reconstitution device with a proximally facing piercer;

FIG. 23 depicts a cross sectional elevation view the reconstitution device of FIG. 22 with a plunger advanced;

FIG. 24 depicts a cross sectional elevation view the reconstitution device of FIG. 22 with a plunger more fully advanced;

FIG. 25 depicts a cross sectional elevation view of another alternative exemplary reconstitution device with a screwing mechanism for dislodging a stopper;

FIG. 26 depicts a cross sectional elevation view the reconstitution device of FIG. 25 with the stopper dislodged;

FIG. 27 depicts a cross sectional elevation view the reconstitution device of FIG. 25 with the stopper dislodged and the plunger advanced;

FIG. 28 depicts a cross sectional elevation view the reconstitution device of FIG. 25 with the stopper dislodged and the plunger more fully advanced;

FIG. 29 depicts a cross sectional elevation view the reconstitution device of FIG. 25 with the stopper dislodged and the plunger fully advanced;

FIG. 30 depicts a cross sectional elevation view of another alternative exemplary reconstitution device with a conventional syringe;

FIG. 31 depicts a cross sectional elevation view the reconstitution device of FIG. 30 with the reconstitution device added to the syringe;

FIG. 32 depicts a cross sectional elevation view the reconstitution device of FIG. 30 with a diluent syringe connected to the reconstitution device;

FIG. 33 depicts a cross sectional elevation view the reconstitution device of FIG. 30 with a diluent syringe injecting a fluid into the reconstitution device;

FIG. 34 depicts a cross sectional elevation view the reconstitution device of FIG. 30 with a diluent syringe axially advancing;

FIG. 35 depicts a cross sectional elevation view of another alternative exemplary reconstitution device with a conventional syringe;

FIG. 36 depicts a cross sectional elevation view the reconstitution device of FIG. 35 with a peg dislodged;

FIG. 37 depicts a cross sectional elevation view the reconstitution device of FIG. 35 with a diluent syringe advanced;

FIG. 38 depicts a cross sectional elevation view of another alternative exemplary reconstitution device with a screwed mating portion;

FIG. 39 depicts a cross sectional elevation view of another alternative exemplary reconstitution device with a screwed mating portion having a reversed orientation;

FIG. 40 depicts a cross sectional elevation view of another alternative exemplary reconstitution device with an elongated bypass; and

FIG. 41 depicts a cross sectional elevation view of another alternative exemplary reconstitution device having two bypasses.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Generally speaking, the following exemplary versions of the reconstitution device are operable to reconstitute a lyophilized material. It will be understood that the lyophilized material may include material that has previously been lyophilized separately from the reconstitution device or in some versions, the material may be lyophilized in a same or associated container with the reconstitution device. In particular, as will be seen in some of the exemplary versions described below, in some cases a liquid or other suitable substance may be controlled using the reconstitution device to combine the liquid with the lyophilized substance to produce the medicament for use with a syringe or other suitable medical device. It will be appreciated that the reconstitution device may be used in some scenarios with an existing syringe. In other scenarios, the reconstitution device may be incorporated with a syringe or syringe-like components such that the reconstitution may be combined with the delivery of the reconstituted material in a single easy to use device.

It will be appreciated that numerous variations of the reconstitution device have been contemplated herein. Indeed, in light of the descriptions set forth below, other exemplary variations will be apparent to one of ordinary skill in view of the teachings herein. Such ways may relate generally to having a diluent chamber separated from a reconstitution chamber by a barrier where the reconstitution chamber contains a lyophilized material. Once the barrier is removed, a diluent contained in the diluent chamber combines with the lyophilized material to reconstitute the lyophilized material.

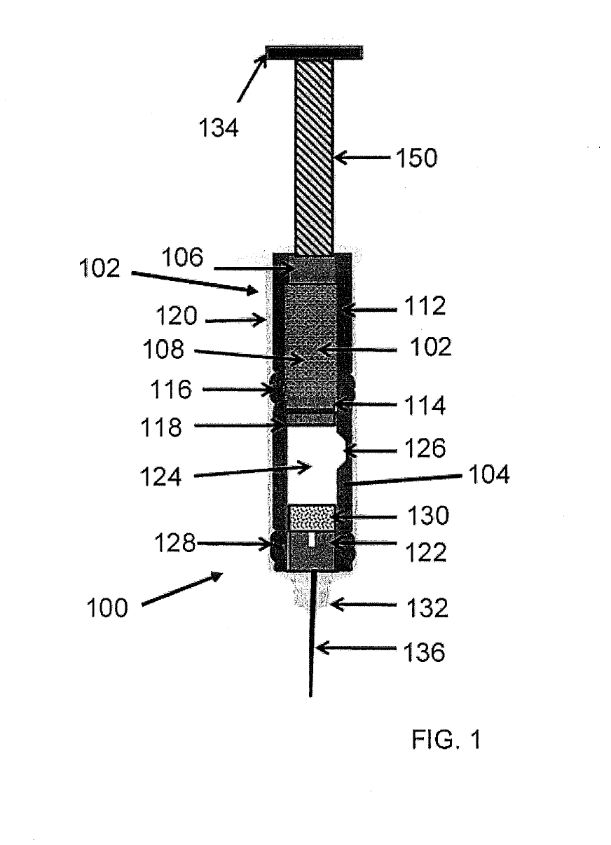

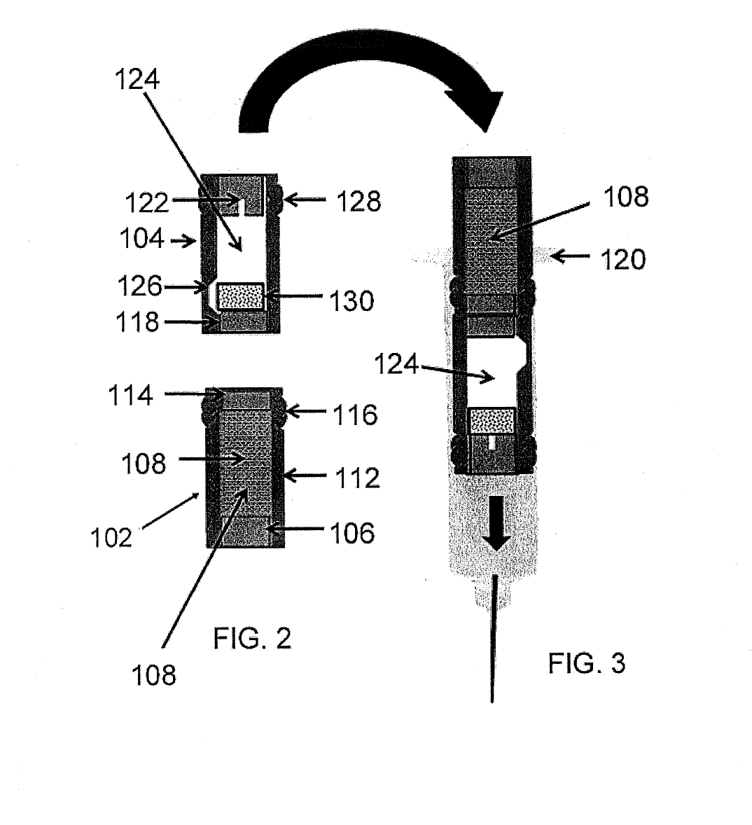

FIG. 1 shows an exemplary reconstitution device (100) within a syringe (120) and having a plunger (150) connected to reconstitution device (100). Reconstitution device (100) includes two chambers: a diluent chamber (102) and a reconstitution chamber (104). Diluent chamber (102) and reconstitution chamber (104) are able to fit together as shown in FIG. 1. FIG. 2 shows diluent chamber (102) and reconstitution chamber (104) separated where FIG. 3 depicts diluent chamber (102) and reconstitution chamber (104) combined and inserted into syringe (120).

Diluent chamber (102) and reconstitution chamber (104) may connect in any suitable manner. For instance, diluent chamber (102) and reconstitution chamber (104) may snap together, may comprise mating screw threading such that diluent and reconstitution chamber (102, 104) may be screwed in together, may be attached using a snap fitting, may be adhered together using friction or adhesive, or may be connected in any other suitable way as would be apparent to one of ordinary skill in the art in view of the teachings herein. Furthermore, in some versions, diluent and reconstitution chambers (102, 104) may be configured such that they are merely placed adjacent to each other within syringe (120) such that diluent and reconstitution chambers (102, 104) are aligned as seen or similarly to FIG. 1 or 3.

Diluent chamber (102) includes a cartridge body (112) containing a diluent (108). The ends of diluent chamber (102) comprise a plunger stopper (106) and on the opposing end a first cartridge stopper (114). Plunger stopper (106) is operable to receive or communicate with plunger (150), which will be described in further detail below. Cartridge body (112) has a cylindrical shape in the illustrated version, but it will be appreciated that cartridge body (112) may have any suitable shape as would be apparent to one of ordinary skill in the art in view of the teachings herein.

Diluent (108) may include any suitable diluent for enabling the reconstitution of a lyophilized material (130) within reconstitution chamber (104). It will be appreciated that diluent (108) may be injected into diluent chamber (102) or alternatively, diluent chamber (102) may be constructed such that diluent (108) is prefilled within diluent chamber (102) prior to delivery to a user.

Plunger stopper (106) is in communication with plunger (150) such that as plunger (150) is advanced axially along diluent chamber (102), plunger stopper (106) also axially advances. Plunger stopper (106) is operable to form a fluid tight, yet movable, seal with diluent chamber (102). While in the illustrated version, plunger stopper (106) and plunger (150) are shown as separate components, it will be appreciated that they may be integrally formed, or otherwise integrated into a single structure. Other suitable variations will be apparent to one of ordinary skill in the art in view of the teachings herein.

First cartridge stopper (114) is positioned within diluent chamber (102) at an end opposing plunger stopper (106). First cartridge stopper (114) forms a fluid tight seal with diluent chamber (102). Furthermore, first cartridge stopper (114) is operable to advance axially from diluent chamber (102) into reconstitution chamber (104) as will be described in further detail below. While the exemplary version shows first cartridge stopper (114) having a thin, cylindrical shape, it will be appreciated that first cartridge stopper (114) may have any suitable shape as would be apparent to one of ordinary skill in the art. For instance, first cartridge stopper (114) may have a thicker cylindrical shape, a ball-like shape, or any other shape suitable to seal an opening. Diluent chamber (102) of the illustrated version has a uniform thickness throughout, but it will be understood that diluent chamber (102) may have any suitable shape as would be apparent to one of ordinary skill in the art in view of the teachings herein.

Diluent chamber (102) further includes one or more o-rings (116) operable to be placed around diluent chamber (102) as seen in FIG. 1 such that diluent chamber (102) can be tightly fit within syringe (120). While the exemplary version shows the use of two o-rings (116) it will be appreciated that any suitable number of o-rings (116) may be used. By way of example only, a single o-ring (116) or three or more o-rings (116) may be used as would be apparent to one of ordinary skill in the art in view of the teachings herein.

Reconstitution chamber (104) includes a second cartridge stopper (118), a needle stopper (122), a reconstitution cavity (124), a bypass (126), lyophilized material (130), and o-rings (128). Second cartridge stopper (118) is positioned on one end of reconstitution chamber (104), and needle stopper (122) is positioned on an opposing end as shown in FIG. 1. Second cartridge stopper (118) forms a fluid tight seal with reconstitution chamber (104) while also being able to advance axially within reconstitution chamber (104). In particular, second cartridge stopper (118) along with first cartridge stopper (114) are operable to advance through reconstitution chamber (104) in response to plunger (150) advancing axially within syringe (120).

As second cartridge stopper (118) advances within reconstitution chamber (104), second cartridge stopper (118) moves across bypass (126), at which point, second cartridge stopper (118) no longer forms a fluid tight seal with reconstitution chamber (104). As second cartridge stopper (118) moves across bypass (126), diluent (108) flows through bypass (126) into reconstitution cavity (124) to contact lyophilized material (130). Bypass (126) of the illustrated version is shaped as an angled cut out within reconstitution chamber (104), but it will be understood that bypass (126) may have any shape suitable to allow diluents (108) to flow around stoppers (114, 118) into reconstitution cavity (124). For instance, bypass (126) may have a curved shape, tunnel shape, or any other suitable shape as would be apparent to one of ordinary skill in the art in view of the teachings herein.

It will be understood that lyophilized material (130) may be any suitable material or medicament that has been lyophilized. In some versions, lyophilized material (130) may include a material lyophilized within reconstitution chamber (104) or lyophilized in a separate location and subsequently added to reconstitution chamber (104). Indeed, lyophilized material (130) may include any suitable material that may require reconstitution prior to use as would be apparent to one of ordinary skill in the art in view of the teachings herein.

Upon passing bypass (126), second cartridge stopper (118) re-engages reconstitution chamber (104) such that second cartridge stopper (118) is operable to force out lyophilized material (130) that has been reconstituted with diluent (108) out of syringe (120) and out through a needle (136). In particular, syringe (120) includes a Luer lock (132) operable to couple with any suitable needle (136). Furthermore, needle (136) is in communication with needle stopper (122) which provides fluid communication between needle (136) and reconstitution cavity (124). Needle stopper (122) in some versions may further be configured to provide an air release such that as plunger (150) advances within syringe (120), an appropriate amount of air is released such that plunger (150) can continue to axially advance within syringe (120).

Plunger (150) includes a top (134) such that the user may be able to grip top (134) to advance plunger (150) within syringe (120). In some version, it will be appreciated that top (134) may be engaged by a machine or other external mechanism operable to engage top (134). Other suitable variations will be apparent to one of ordinary skill in the art in view of the teachings herein.

Reconstitution chamber (104) is placed within syringe (120) and retained within syringe (120) using o-rings (128). While the illustrated version uses o-rings (128) to retain reconstitution chamber (104), it will be appreciated that other suitable ways of retaining reconstitution chamber (104) within syringe (120) may be used. For instance, an adhesive, glue, any number of o-rings, clips, latches, or any suitable structure may be used as would be apparent to one of ordinary skill in the art in view of the teachings herein. It will be further understood that diluent chamber (102) may be retained within syringe (120) in a substantially similar manner using o-rings (116) or any other suitable structure operable to retain or provide sufficient friction between diluent chamber (102) and syringe (120).

FIG. 2 shows diluent chamber (102) and reconstitution chamber (104) separated. It will be understood that, for instance, diluent (108) may be injected into diluent chamber (102) prior to connecting diluent chamber (102) and reconstitution chamber (104). Furthermore, lyophilized material (130) may be inserted into reconstitution chamber (104) prior to connecting diluent chamber (102) and reconstitution chamber (104) as well. Diluent chamber (102) and reconstitution chamber (104) may further be shipped, stored, or otherwise maintained separately until reconstitution device (100) is ready for use.

In one exemplary version of using reconstitution device (100), diluent chamber (102) and reconstitution chamber (104) may be loaded into syringe (120). It will be understood that luer lock (132) portion of syringe (120) may be used to vent air as diluent chamber (102) and reconstitution chamber (104) are being inserted into syringe (120).

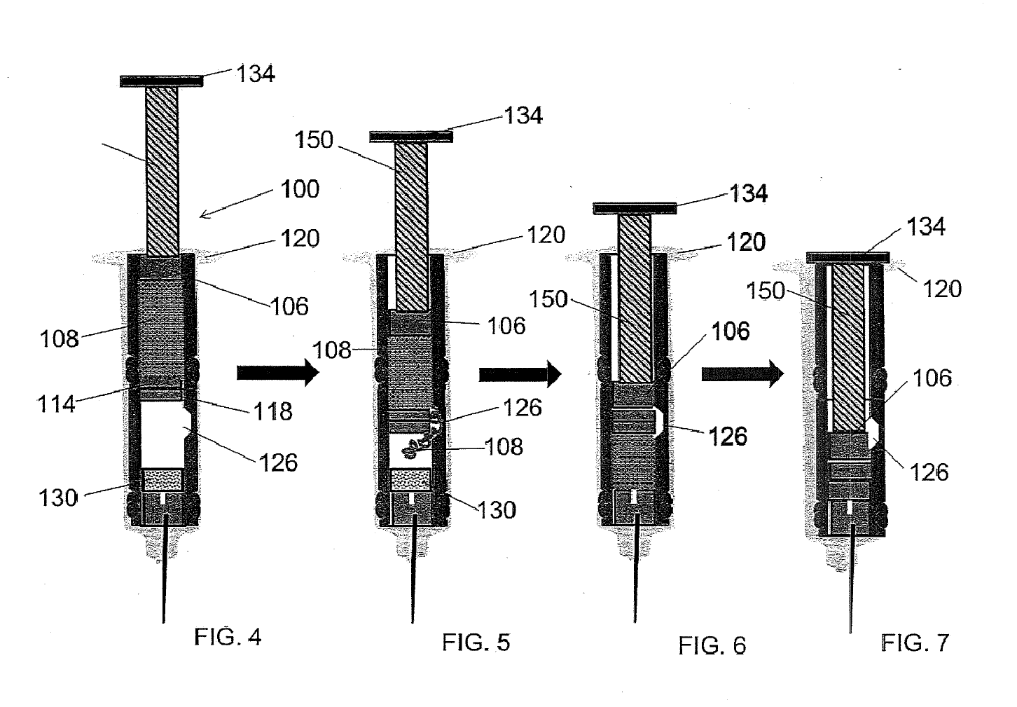

FIGS. 4-7 illustrate an exemplary operation sequence of reconstitution device (100). Once ready for use, FIG. 4 shows reconstitution device (100) ready for use with diluent chamber (102) positioned on top of reconstitution chamber (104). Plunger (150) is advanced in FIG. 5. As user presses top (134), plunger (150) and plunger stopper (106) advance axially within diluent chamber (102). As a result, diluent (108), first cartridge stopper (114) and second cartridge stopper (118) also advance within diluent chamber (102). First cartridge stopper (114) and second cartridge stopper (118) advance past bypass (126), wherein after diluent (108) flows from diluent chamber (102) into reconstitution chamber (104). Upon contact between diluent (108) and lyophilized material (130), lyophilized material (130) begins to reconstitute. It will further be understood that as more diluent (108) is added to lyophilized material (130), lyophilized material (130) may become more fully reconstituted as shown in FIG. 6 where lyophilized material (130) has fully reconstituted. Finally, reconstituted lyophilized material (130) may then be urged out of syringe (120) by further advancing plunger (150) as shown in FIG. 7. As plunger (150) advances, reconstituted material exits syringe (120) through needle (136). It will be appreciated that while the illustrated version shows one use of reconstitution device (100), other variations will be apparent to one of ordinary skill in the art in view of the teachings herein. For instance, rather than being actuated by hand, reconstitution device (100) may be controlled by a machine or other suitable device.

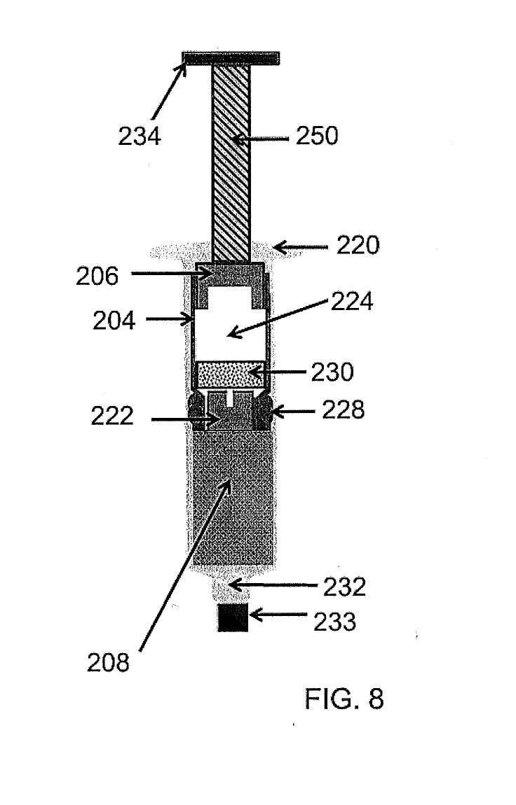

FIG. 8 shows another exemplary version of a reconstitution device (200) having a syringe (220) and a plunger (250). In the exemplary version, a diluent (208) is contained within syringe (220). While in the illustrated version, diluent (208) may be contained within syringe (220) directly, it will be appreciated that diluent (208) could be in another container within syringe (220) or having any other suitable configuration as would be apparent to one of ordinary skill in the art.

While diluent (208) is stored within syringe (220), a plug (233) covers luer lock portion (232) of syringe (220) such that diluent (208) does not inadvertently leak from syringe (220). While the exemplary version depicts plug (233) as being used to cover luer lock (232) portion, it will be appreciated that any suitable means for preventing diluent (208) from unintentionally leaking from syringe (220) may be used.

In the illustrated version, reconstitution device (200) further comprises a reconstitution chamber (204) operable to be inserted into syringe (220) and held within syringe (220) through the use of o-rings (228). While the illustrated version shows o-rings (228) positioned near diluent (208), it will be understood that o-rings (228) may be positioned at any suitable region along reconstitution chamber (204) as would be apparent to one of ordinary skill in the art. Furthermore, while the exemplary version includes two o-rings (228), it will be understood that any suitable number of o-rings (228) may be used as would be apparent to one of ordinary skill in the art.

Reconstitution chamber (204) includes a stopper (222) positioned at an end of reconstitution chamber (204) and adjacent to diluent (208). Stopper (222) is operable to be displaced towards plunger (250) as plunger (250) advances through reconstitution chamber (204) as will be described in further detail below. Reconstitution chamber (204) further includes plunger stopper (206), which is connected with plunger (250). Plunger (250) is advanced by a user or machine applying axial pressure to top (234).

Plunger stopper (206) of the exemplary version includes a portion of the center of plunger stopper (206) cut out, but other suitable configurations for plunger stopper (206) may be used as would be apparent to one of ordinary skill in the art in view of the teachings herein. For example, plunger stopper (206) may simply comprise a cylindrical plug operable to maintain a fluid tight seal against the walls of reconstitution chamber (204) while advancing axially within reconstitution chamber (204).

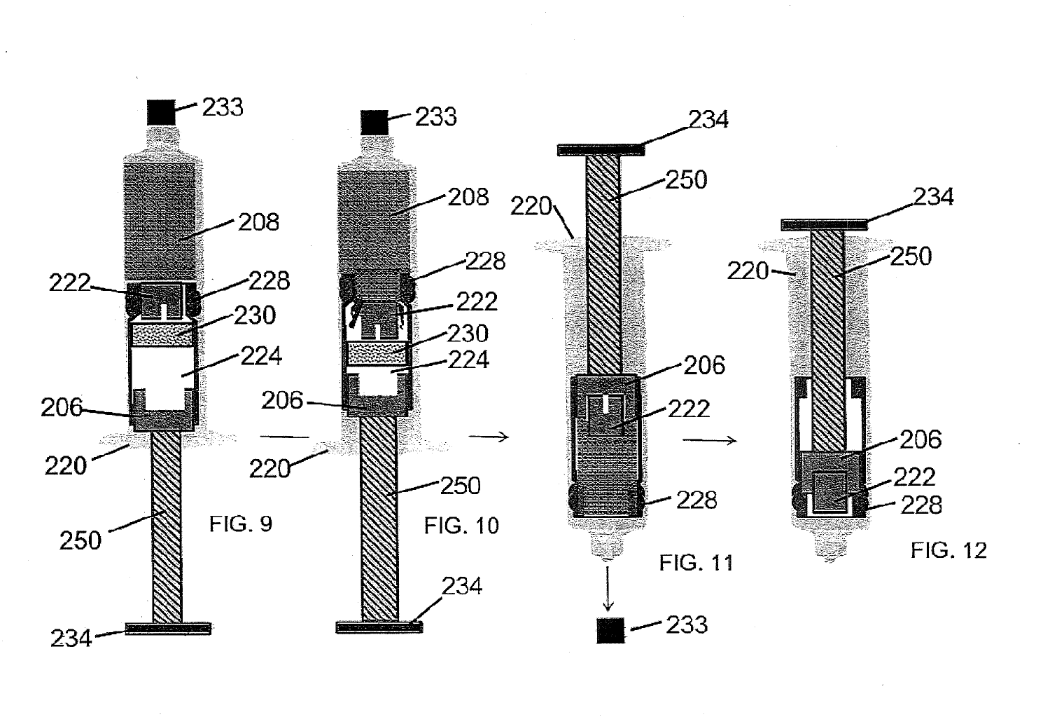

In the illustrated version, FIGS. 9-12 show an exemplary sequence for using reconstitution device (200). FIG. 9 shows reconstitution device (200) being ready for use having diluent (208) contained within syringe, which will facilitate reconstitution of a lyophilized material (230) within reconstitution chamber (204). It will be understood that lyophilized material (230) may be added to reconstitution chamber (204). In some versions, it will be understood that lyophilized material (230) may be lyophilized within reconstitution chamber (204). For instance, liquid medicament may be added to reconstitution chamber (204) followed by lyophilizing the liquid medicament to form lyophilized material (230). Thereafter, reconstitution chamber (204) may be stored until ready for use.

Once the user is ready to reconstitute lyophilized material (230), the user axially advances plunger (250) by pressing on top (234). As top (234) is pressed, plunger (250) and plunger stopper (206) attempt to advance within reconstitution chamber (204). In this embodiment, stopper (222) is engaged within reconstitution chamber (204) such that axial forces may displace stopper (222). In particular, o-rings (228) maintain reconstitution chamber (204) at one position axially within syringe (220). The frictional forces keeping o-rings (228) with reconstitution chamber (204) in place are stronger than the frictional forces keeping stopper (222) in place. As a result, at some point as plunger (250) is advanced, stopper (222) is displaced towards plunger (250) as shown in FIG. 10. As stopper (222) is displaced, diluent (208) enters reconstitution chamber (204), also shown in FIG. 10.

Upon diluent (208) contacting lyophilized material (230), reconstitution of lyophilized material (230) begins and continues until material (230) is fully reconstituted as shown in FIG. 11. Furthermore, reconstitution chamber (204) is also axially advanced within syringe (220). It will also be understood that as reconstitution chamber (204) moves axially within syringe (220), reconstitution chamber (220) is configured to form a fluid tight seal with the walls of syringe (220). The user may then remove plug (233) in order to prepare syringe (220) for use. As shown in FIG. 12, plunger (250) may be further advanced within syringe (220) in order to urge reconstituted fluid from syringe (220). While the illustrated version shows syringe (220) without a needle at Luer lock (232), it will be appreciated that syringe (220) may be used with a needle as would be understood by one of ordinary skill in the art in view of the teachings herein.

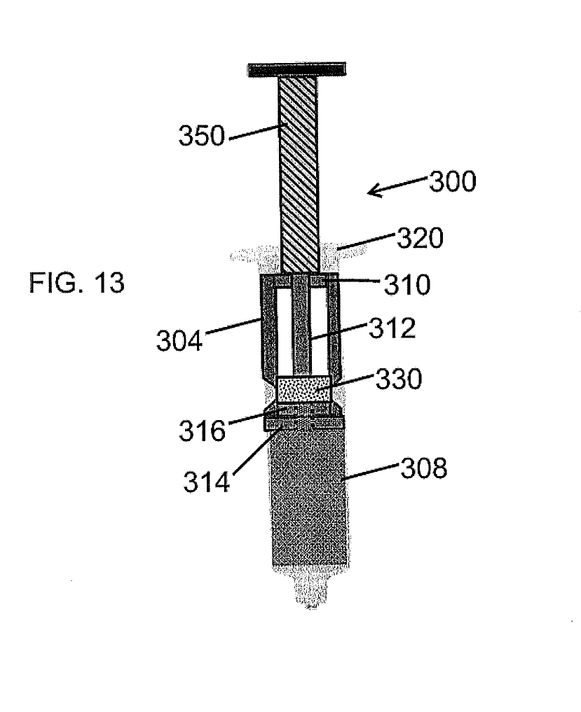

FIG. 13 shows another exemplary version of a reconstitution device (300) including a syringe (320) and plunger (350). Reconstitution device (300) may be used in a similar manner in that reconstitution device (300) can reconstitute a lyophilized material (330) by providing a mechanism for adding a diluent (308) to lyophilized material (330).

Reconstitution device (300) includes a reconstitution chamber (304) having a proximal cap (310) and a rod (312) attached to proximal cap (310). Rod (312) may engage proximal cap (310) through threading coupled with a hole extending through the center of proximal cap (310), but it will be understood that rod (312) may couple with proximal cap (310) in any suitable manner. For instance, proximal cap (310) and rod (312) may be coupled through a friction fit or coupled in any other manner as would be apparent to one of ordinary skill in the art in view of the teachings herein. As shown, proximal cap (310) comprises a circular plate operable to couple with reconstitution chamber (304).

Rod (312) is further coupled with plunger (350) such that as plunger (350) is rotated, rod (312) retracts in the direction of plunger (350), which will be discussed in further detail below in relation to reconstitution of lyophilized material (330). Reconstitution chamber (304) further includes an outer plate (314) and an inner plate (316). Outer plate (314) and inner plate (316) form an end of reconstitution chamber (304) opposing proximal cap (310). Inner plate (316) and outer plate (314) include threading to couple with rod (312) such that rod (312) within reconstitution chamber (304) can retain inner plate (316) and outer plate (314) at end of reconstitution chamber (304). Rod (312) extends through inner plate (316) and outer plate (314) as well as through lyophilized material (330). Inner plate (316) and outer plate (314) are coupled to reconstitution chamber (304) such that as rod (312) rotates, rod (312) may be removed from inner plate (316) and outer plate (314).

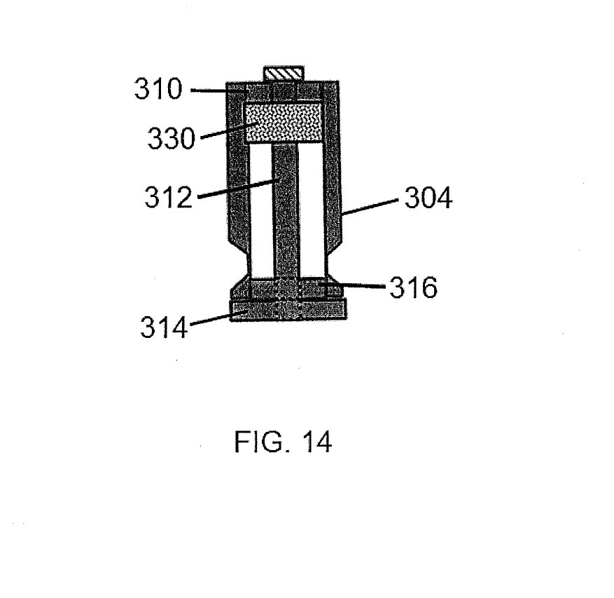

FIG. 14 shows reconstitution chamber (304) prior to being placed in syringe (320). As can be seen, reconstitution chamber (304) may be shipped separately or may be shipped with syringe (320). As described above, FIG. 13 of course shows reconstitution chamber (304) inserted within syringe (320). It will be appreciated that syringe (320) may comprise a conventional syringe or one made specifically for use with reconstitution chamber (304).

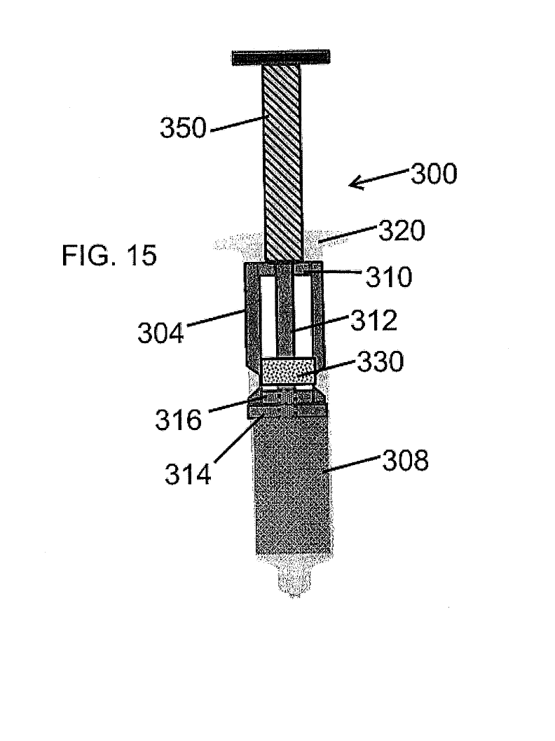

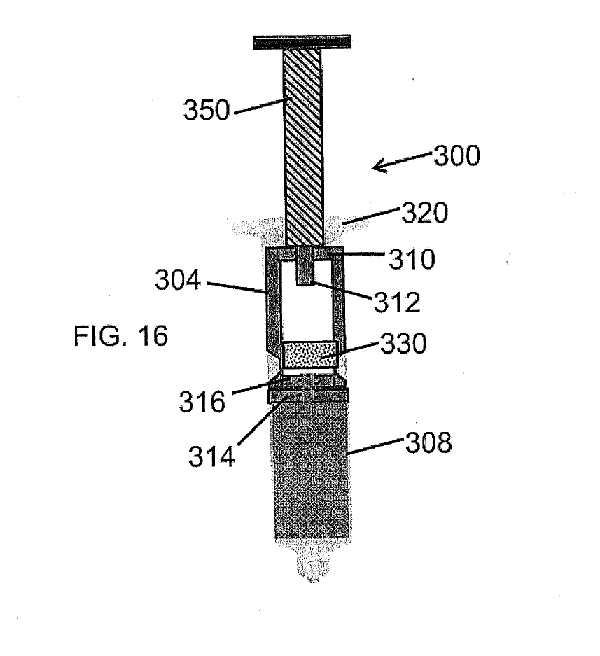

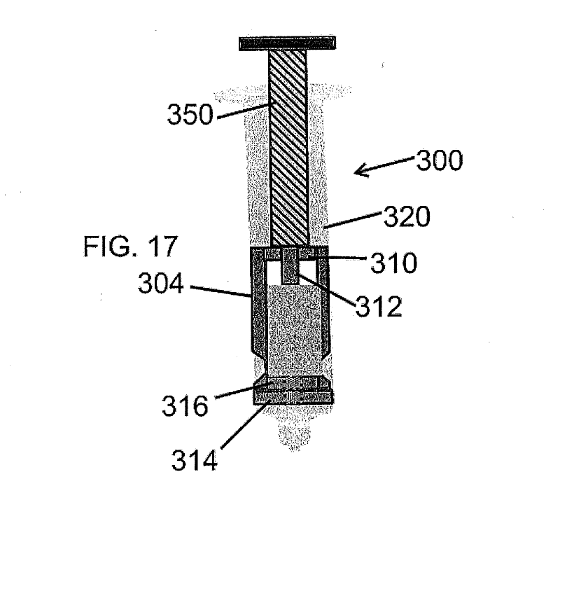

Once the user is ready to reconstitute lyophilized material (330), the user may rotate plunger (350) within reconstitution chamber (304) such that rod (312) retracts into plunger (350) as seen in FIG. 15. Proximal cap (310) is configured to remain stationary such that as the user rotates plunger (350) with plunger (350) abutting proximal cap (310), rod (312) is drawn into plunger (350). As a result, rod (312) dislodges from inner plate (316) and outer plate (314). FIG. 16 shows rod (312) being almost fully retracted into plunger (350). Diluent (308) then flows into reconstitution chamber (304) as shown in FIG. 17. It will be understood that lyophilized material (330) may be reconstituted through convection or may also be assisted by the user shaking or otherwise moving reconstitution chamber (304). Once lyophilized material (330) has been reconstituted, reconstitution device (300) may be set aside or may be ready to use. As also seen in FIG. 17, as plunger (350) is advanced, reconstitution chamber (304) may advance to further allow diluent (308) to completely flow into reconstitution chamber (304).

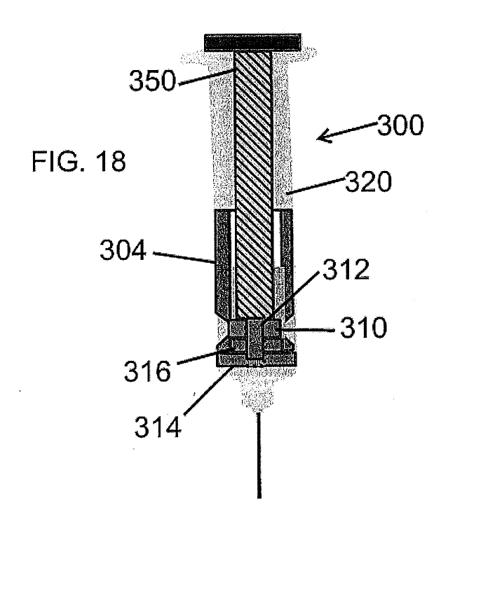

When the user is ready to inject the reconstituted fluid, the user may further axially advance plunger (350) as shown in FIG. 18, which causes proximal cap (310) to advance and urge fluid from reconstitution device (300). While the illustrated version shows one exemplary method of using reconstitution device (300), it will be understood that other suitable ways of using reconstitution device (300) may be used as would be apparent to one of ordinary skill in the art in view of the teachings herein.

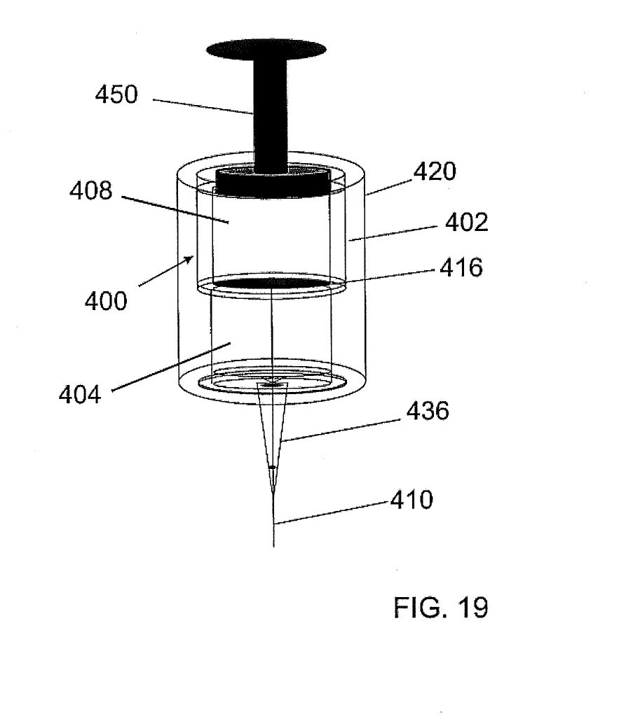

FIG. 19 depicts yet another alternative exemplary version of a reconstitution device (400) including a syringe (420), plunger (450), and a needle (436) attached to syringe (420). A reconstitution chamber (404) is separated by a disc or stopper (416) positioned between reconstitution chamber (404) and diluent chamber (402). Stopper (416) is configured to form a fluid tight seal between reconstitution chamber (404) and diluent chamber (402). While in the exemplary version, stopper (416) has a flat disc shape, it will be understood that stopper (416) may have any suitable shape or construction as would be apparent to one of ordinary skill in the art in view of the teachings herein. For instance, stopper (416) may include an elongated cylinder, a ball seal, or any other suitable shape.

Stopper (416) is coupled with a thread (410) that extends axially from stopper (416) out needle (436). By way of example, thread (410) may be connected with stopper (416) by tying thread (410) to stopper (416), fusing thread (410) to stopper (416) or any other suitable way of connecting thread (410) with stopper (416). When the user is ready to reconstitute a lyophilized material (not shown) that may be contained, for instance, in reconstitution chamber (404), the user pulls thread (410) such that stopper (416) dislodges thus allowing a diluent (408) to flow towards needle (436), come in contact with a lyophilized material, and reconstitute the lyophilized material. Thereafter, plunger (450) may be advanced within syringe (420) in order to deliver the reconstituted material from syringe (420).

It will also be understood that while the illustrated version uses thread (410) to dislodge stopper (416), other suitable variations may be used as would be apparent to one of ordinary skill in the art in view of the teachings herein. For instance, thread (410) may comprise a wire or thin post extending through needle (436). It will be understood that thread (410) may be constructed of generally an inert material such that a diluent or reconstituted material does not react with thread (410).

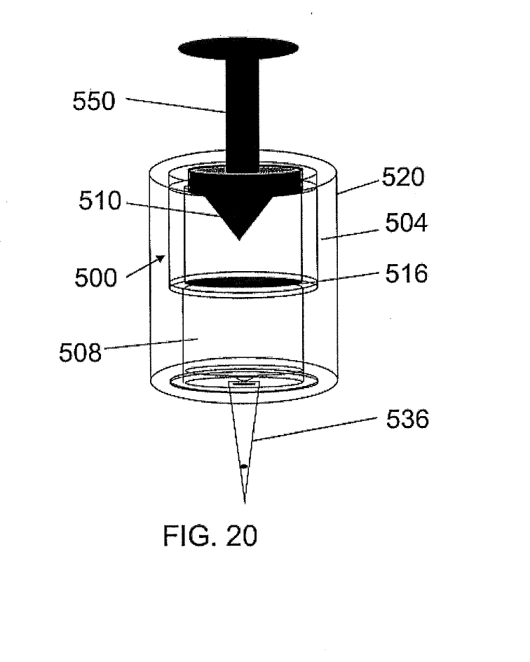

FIG. 20 shows yet another exemplary variation of a reconstitution device (500) for use with a syringe (520) where a cone (510) or other suitable piercing structure is in communication with plunger (550). Reconstitution device (500) further includes a plate (516) separating reconstitution chamber (504) and diluent (508) contained within syringe (520). Reconstitution chamber (504) is configured to hold a lyophilized material (not shown) capable of being reconstituted to form a usable fluid or medicament.

Plate (516) is configured to provide a fluid seal between reconstitution chamber (504) and diluent (508). While reconstitution chamber (504) and diluent (508) are positioned in the illustrated version such that reconstitution chamber (504) is axially above diluent (508) it will be understood that diluent (508) may instead be positioned as the upper chamber and reconstitution chamber (504) may be positioned below diluent (508).

When the user desires to reconstitute a lyophilized material contained in reconstitution chamber (504), the user may advance plunger (550) such that cone (510) pierces plate (516). Cone (510) includes a sharpened end such that cone (510) can pierce plate (516) without excessive force. It will further be understood that cone (510) may be operable to displace plate (516) rather than pierce it. Upon breaching plate (516), diluent (508) flows into reconstitution chamber (504) such that diluent (508) is able to reconstitute any lyophilized material contained within reconstitution chamber (504). It will be understood that syringe (520) includes a one-way valve or other release mechanism such that sufficient air is released from reconstitution chamber (504) to allow plunger (550) to advance.

While the exemplary version shows cone (510) having a cone or triangular shape, it will be appreciated that any suitable shape may be used. For instance, rather than a cone shape, a pyramid shape, a needle shape, a cylindrical post, a curved rod, a chisel shape, a protrusion having multiple prongs or any other suitable structure may be used as would be apparent to one of ordinary skill in the art in view of the teachings herein. Furthermore, cone (510) in the illustrated version is positioned such that cone (510) is coaxial with plunger (550). However, it will be understood that cone (510) may be positioned at any suitable position. For instance, cone (510) may be positioned offset in relation to plunger (550). Cone (510) may also be positioned near the side wall of syringe (520). Indeed, cone (510) or any suitable piercing or displacement structure may be in communication with plunger (550) at any suitable location to pierce or displace plate (516).

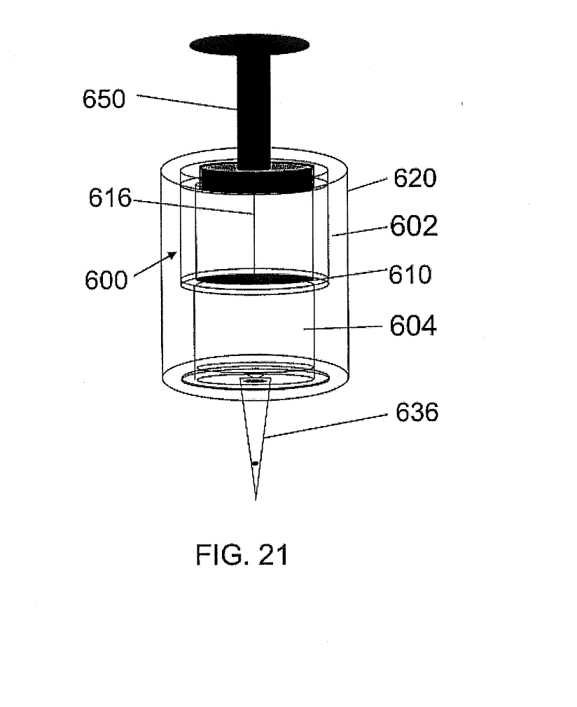

FIG. 21 shows yet another exemplary version of a reconstitution device (600) having a detachable thread (616) for assisting in reconstitution of a lyophilized material within a syringe (620). Thread (616) is in communication with a plunger (650) as well as stopper (610).

It will be understood that thread (616) remains relatively taut. As thread (616) remains taut, it will be understood that stopper (610) forms a fluid seal between reconstitution chamber (604) and diluent chamber (602). As a result, plunger (650) may be pulled away from needle (636) in order to detach thread (616) from plunger (650). When thread (616) detaches from plunger (650), stopper (610) is able to fall or tip such that fluid in diluent chamber (602) can combine with fluid in reconstitution chamber (604), which contains, for instance, a lyophilized material for reconstitution. While in the exemplary version, reconstitution chamber (604) is below diluent chamber (602), it will be understood that any suitable orientation for reconstitution chamber (604) and diluent chamber (602) may be used as would be apparent to one of ordinary skill in the art in view of the teachings herein.

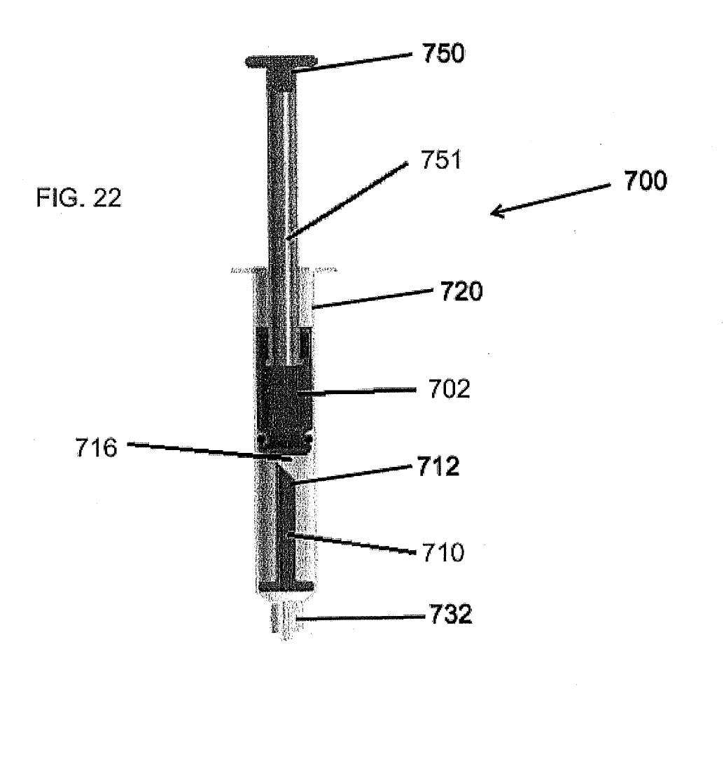

FIG. 22 shows yet another exemplary version of a reconstitution device (700) having a perforator (710) used to break a seal (716) to enable reconstitution. Reconstitution device (700) includes a plunger (750) in a syringe (720). Syringe (720) includes a diluent or fluid chamber (702) where the bottom portion of diluent chamber (702) includes seal (716). In the exemplary version, seal (716) includes a foil seal, but it will be appreciated that seal (716) may include any suitable material capable of being pierced by perforator (710). As a result, as plunger (750) axially advances through syringe (720), it will be understood that piercing tip (712) of perforator (710) breaks seal (716) thereby establishing fluid communication between fluid chamber (702) and syringe (720). Furthermore, plunger (750) includes a hollow region (751) operable to fit perforator (710), which will be discussed in further detail below.

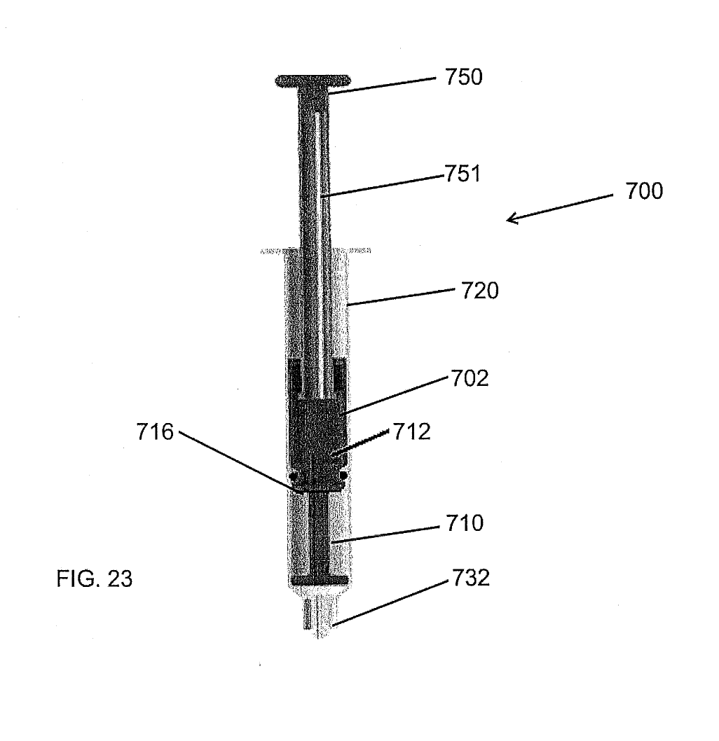

It will be understood that diluent chamber (702) contains a fluid or other suitable diluent operable to reconstitute the lyophilized material (not shown) located, for instance, around perforator (710). As a result, establishing fluid communication by breaking seal (716) will enable a diluent to enter bottom portion of syringe (720) to reconstitute a lyophilized material. For instance, FIG. 23 shows plunger (750) axially advanced within syringe (720). As a result, piercing tip (712) breaks seal (716) and enters diluent chamber (702). Fluid in diluent chamber (702) flows toward perforator (710) and to the extent that syringe (720) contains lyophilized material or other suitable materials to be reconstituted, facilitates reconstitution of such material. It will be understood that as sufficient time elapses, lyophilized material fully reconstitutes or otherwise reconstitutes a sufficient amount such that a user may then decide to inject the reconstituted fluid using plunger (750).

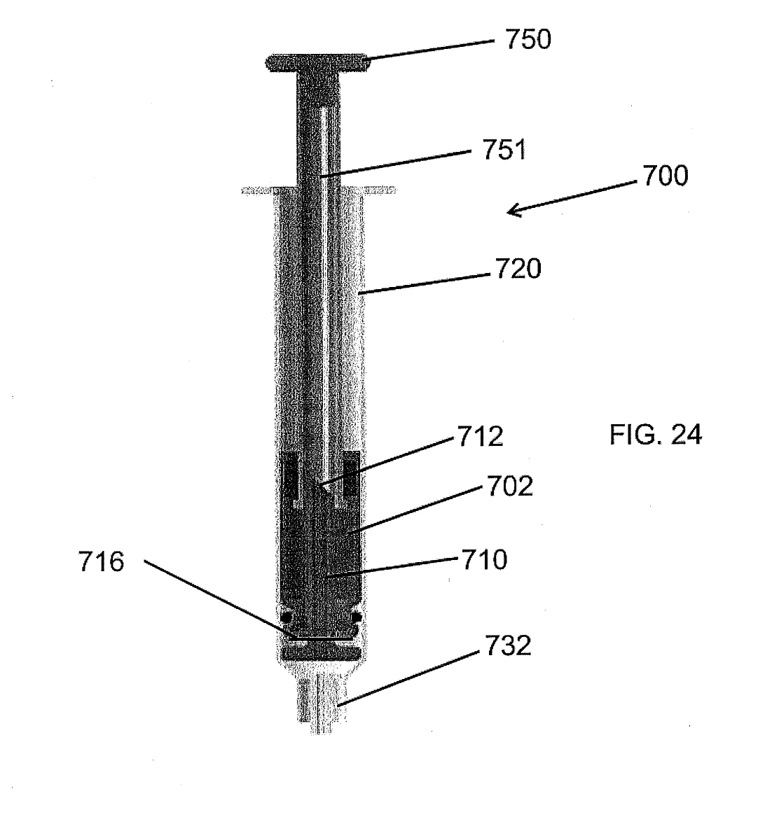

FIG. 24 shows plunger (750) being further advanced such that diluent chamber (702) also axially advances within syringe (720). Piercing tip (712) enters hollow region (751) within plunger (750) in order to provide more room for piercing tip (712). As diluent chamber (702) advances through syringe (720), fluid is urged out through luer lock portion (732). While the exemplary version does not depict a needle attached to luer lock portion (732), it will be understood that luer lock portion (732) may have a needle connected to luer lock portion (732) to deliver the reconstituted fluid through the needle. Furthermore, while the exemplary version shows piercing tip (712) as an angled piercing portion, it will be understood that any suitable tip (712) may have a configuration capable of piercing seal (716).

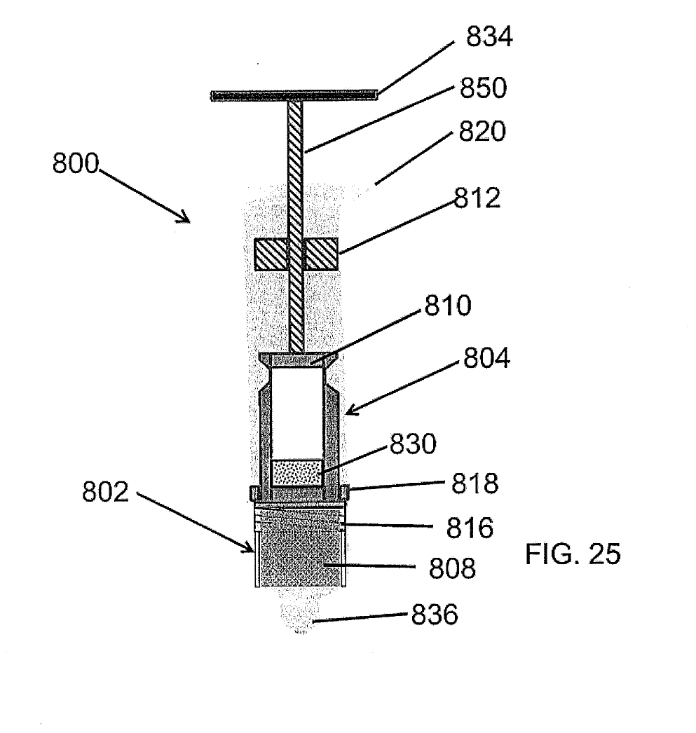

FIG. 25 shows yet another exemplary version of a reconstitution device (800) having an alternative mechanism for reconstituting a lyophilized material (830). Reconstitution device (800) includes a syringe (820), plunger (850), reconstitution chamber (804), and diluent chamber (802). Reconstitution chamber (804) contains a lyophilized material (804) and includes a proximal stopper (810) and distal stopper (818). A syringe stopper (812) holds plunger (850) within syringe (820). The proximal end of plunger (850) includes a handle (834) operable to be rotated, which will be discussed in further detail below.

Diluent chamber (802) includes screw engagement portion (816) and contains a diluent (808). The end of syringe (820) includes a luer lock (836) portion operable to engage, for instance, a needle, or other device able to engage luer lock (836).

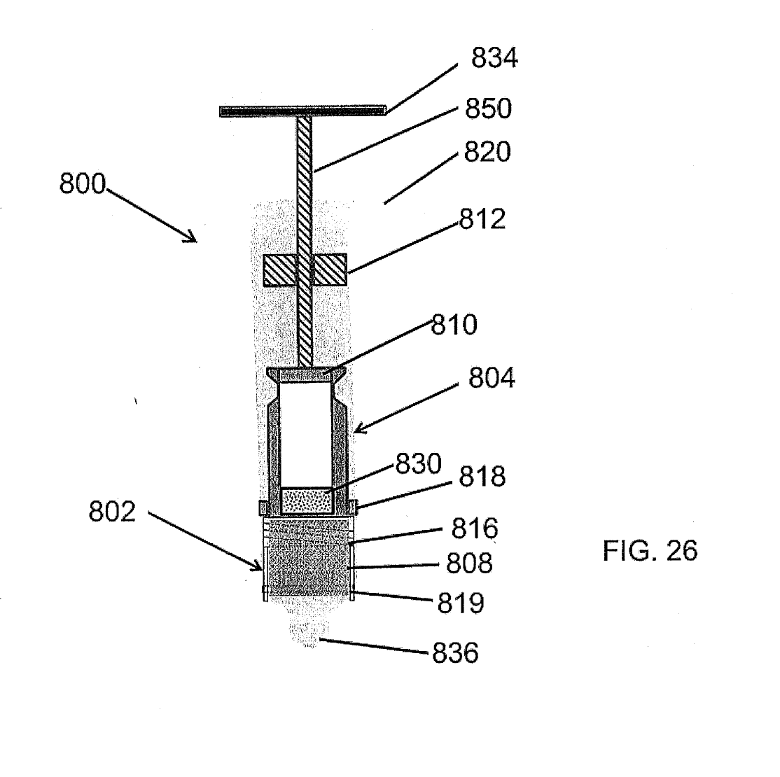

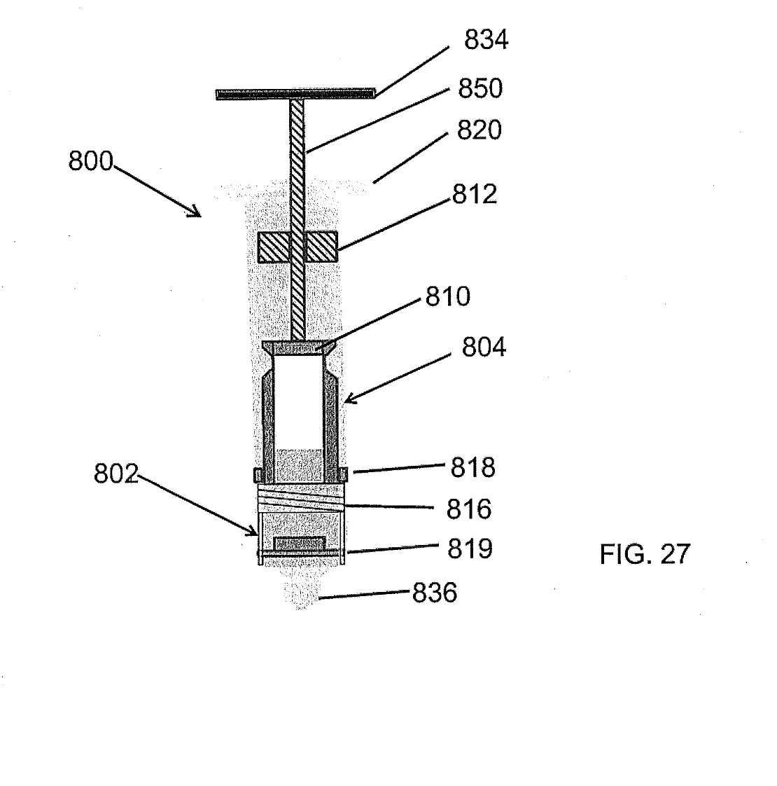

In this embodiment, when the user is ready to reconstitute lyophilized material (830), the user turns diluent chamber (802) or distal stopper (818) to cause diluent plug (819) shown in FIG. 26 to advance and unseal diluent chamber (802) from reconstitution chamber (804) thereby establishing fluid communication between diluent chamber (802) and reconstitution chamber (804). It will be understood that screw engagement portion (816) contains threading to facilitate movement of diluent plug (819) through diluent chamber (802). For instance, diluent plug (819) may have screw threading complementary with screw engagement portion (816). Furthermore, diluent plug (819) may be configured to remain rotationally constant such that rotating screw engagement portion (816) causes diluent plug (819) to advance axially within syringe (850). Other suitable mechanisms for diluent plug (819) to dislodge in response to user rotation of a portion of reconstitution device (800) may be used as would be apparent to one of ordinary skill in the art in view of the teachings herein. As mentioned above, either diluent chamber (802) or distal stopper (818) can be configured to cause movement of diluent plug (819) through diluent chamber (802). As a result of diluent plug (819) advancing, diluent (808) flows into reconstitution chamber (804) to facilitate reconstitution of lyophilized material (830). After sufficient time elapses, lyophilized material (830) is reconstituted as shown in FIG. 27.

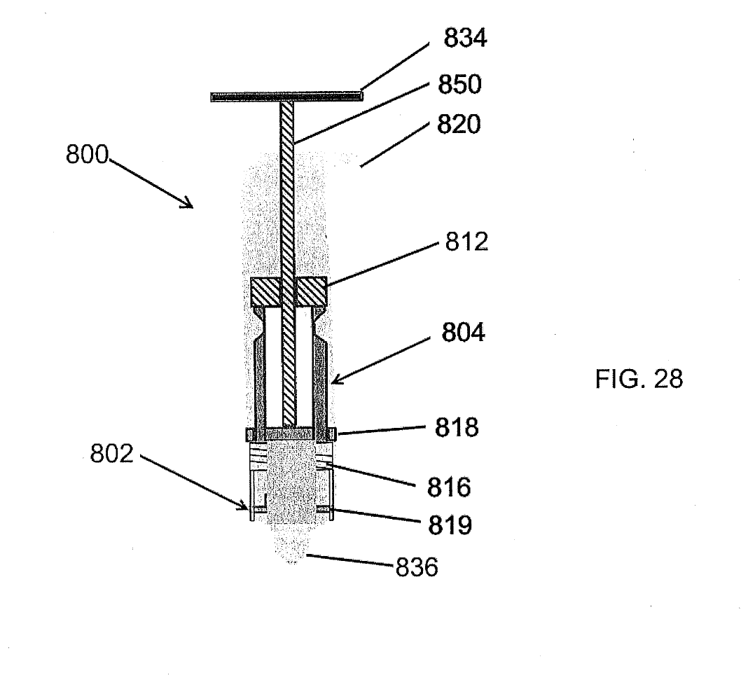

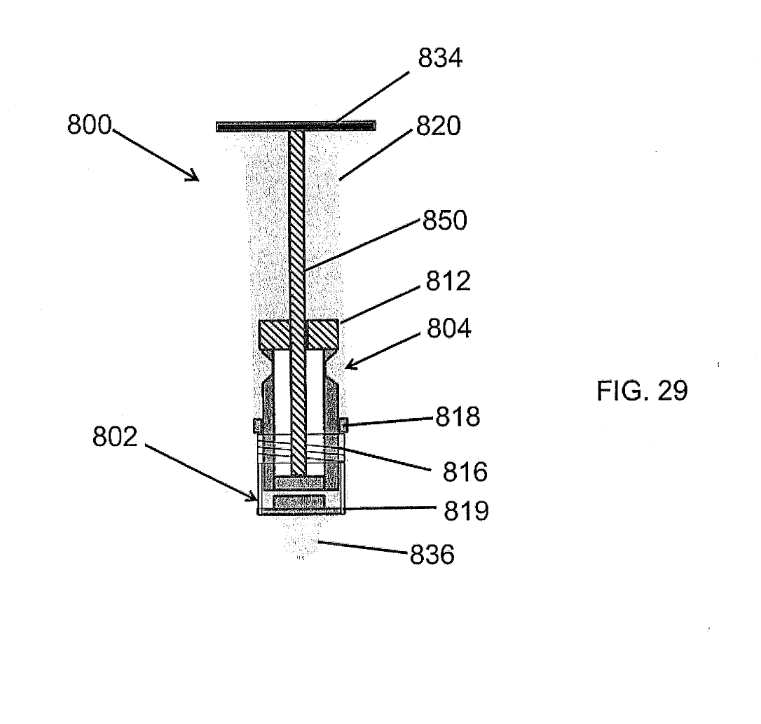

FIG. 28 shows plunger (850) advancing through syringe (820). Syringe stopper (812) contacts reconstitution chamber (804) as plunger (850) moves through syringe (820). As plunger (850) moves through syringe (820), fluid is urged out of syringe (820) through luer lock (836) region. FIG. 29 shows plunger (850) fully advanced, which allows reconstituted fluid to be completely urged from syringe (820).

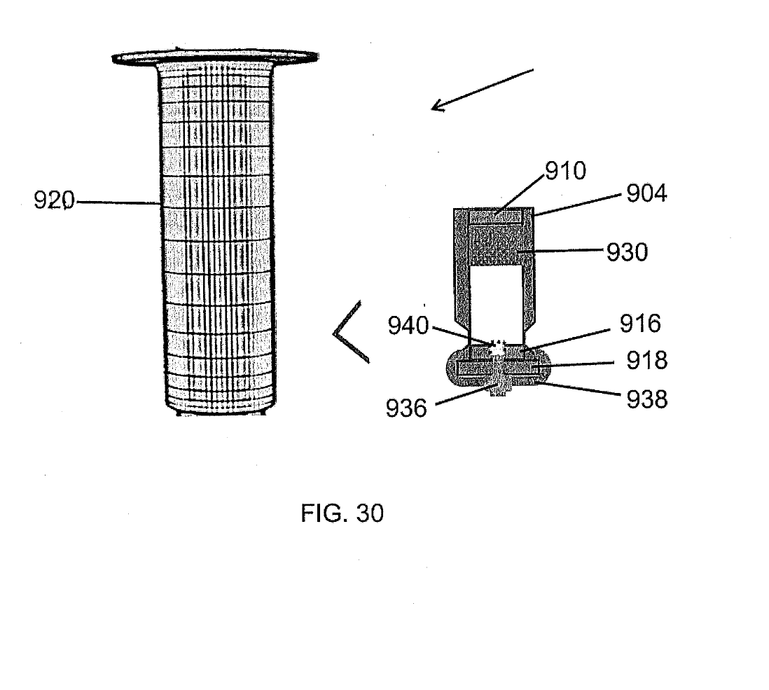

FIG. 30 shows yet another exemplary version of a reconstitution device (900) as might be used with a conventional or off-the-shelf syringe (920). Reconstitution device (900) includes a reconstitution chamber (904) with a proximal stopper (910), lyophilized material (930), an inner stopper (916), outer stopper (918), and a luer lock (936) where luer lock (936) is inserted into an opening (940) extending through inner stopper (916) and outer stopper (918). Reconstitution chamber (904) further includes a crimped portion (938) covering inner stopper (916) and outer stopper (918).

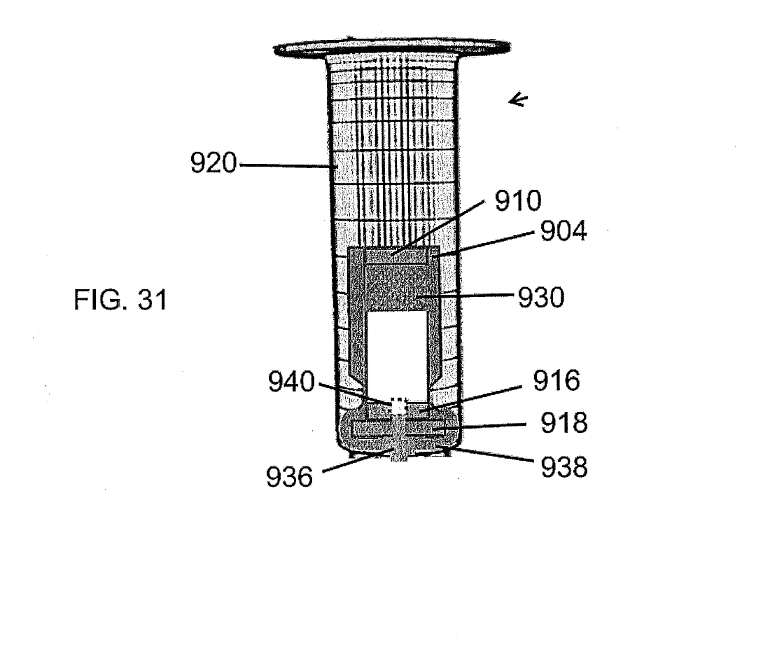

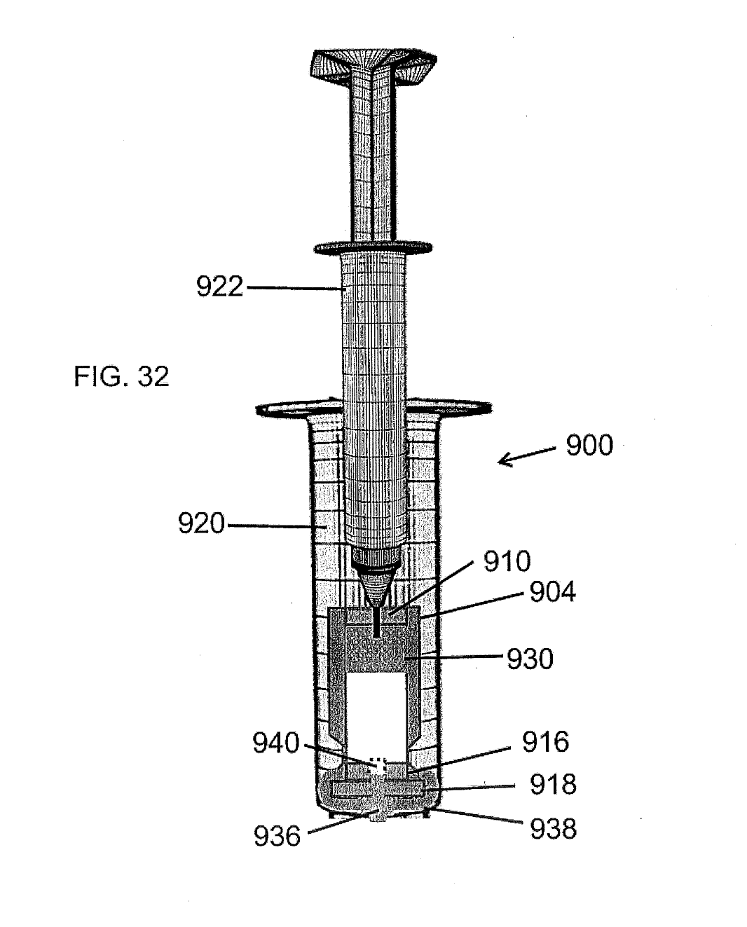

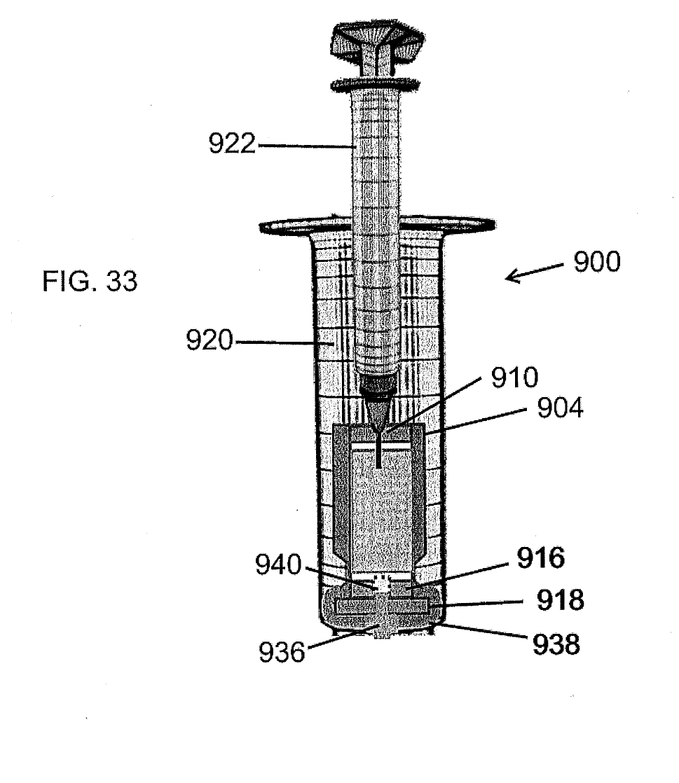

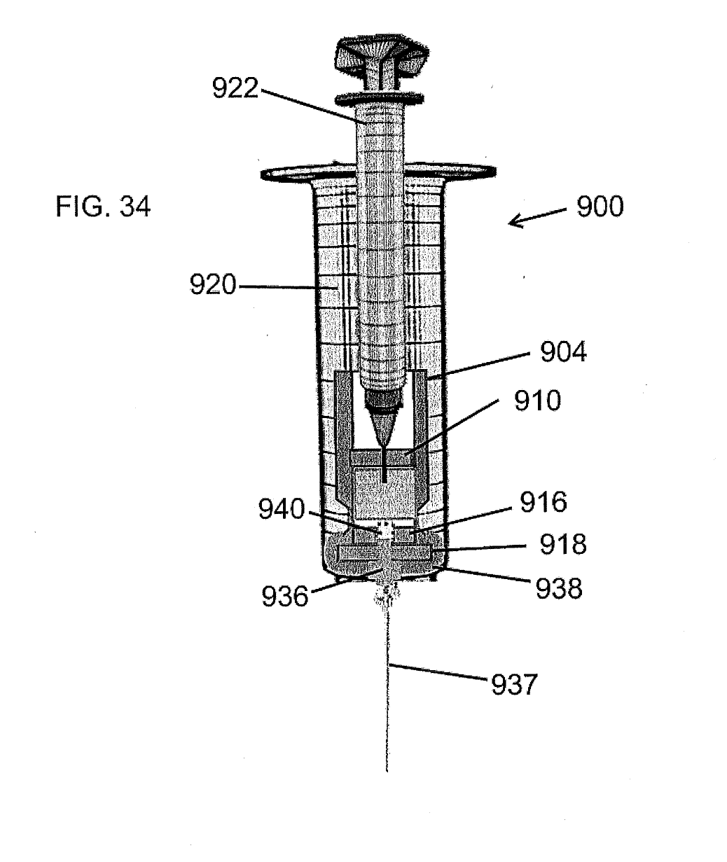

Reconstitution chamber (904) is insertable into syringe (920) as shown in FIG. 31. Crimped portion (938) is operable to help maintain a snug fit within syringe (920). When the user wishes to reconstitute lyophilized material (930), a diluent syringe (922) may be inserted into syringe (920) such that it pierces proximal stopper (910) as seen in FIG. 32. Diluent syringe (922) may then be used to inject a diluent into lyophilized material (930), which reconstitutes lyophilized material (930) as seen in FIG. 33. Thereafter, diluent syringe (922) may be advanced through syringe (920) such that proximal stopper (910) advances through reconstitution chamber (904) and urges fluid out of needle (937) as shown in FIG. 34. It will be understood that inserting diluent syringe (922) into syringe (920) allows diluent syringe (922) to be used operably as a plunger for proximal stopper (910) to axially move proximal stopper (910). As also seen in FIG. 34, diluent syringe (922) includes its own plunger for injecting fluid into reconstitution chamber (904). Diluent syringe (922) in combination with syringe (920) is operable to act as a 3-tier telescoping system.

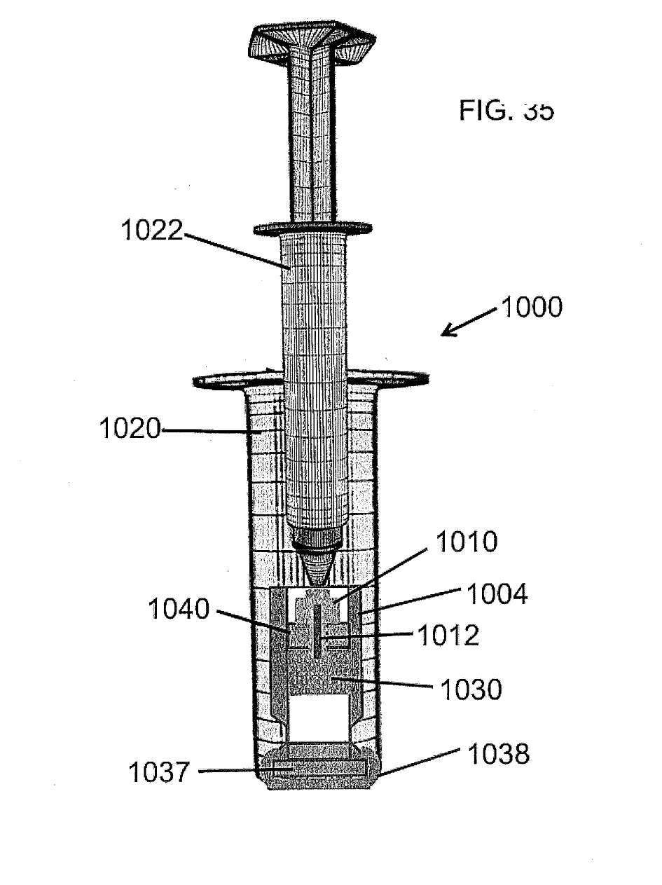

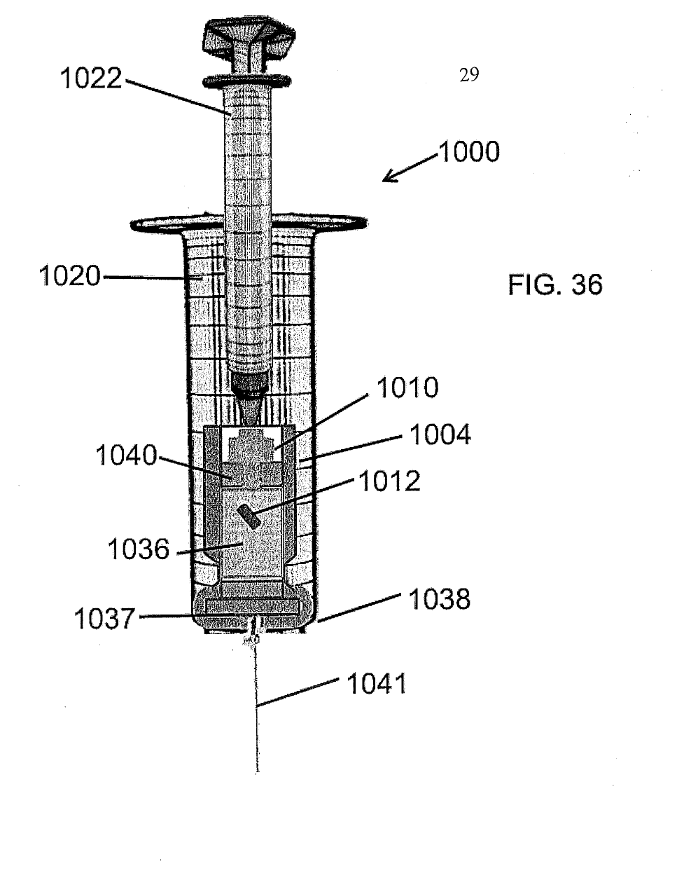

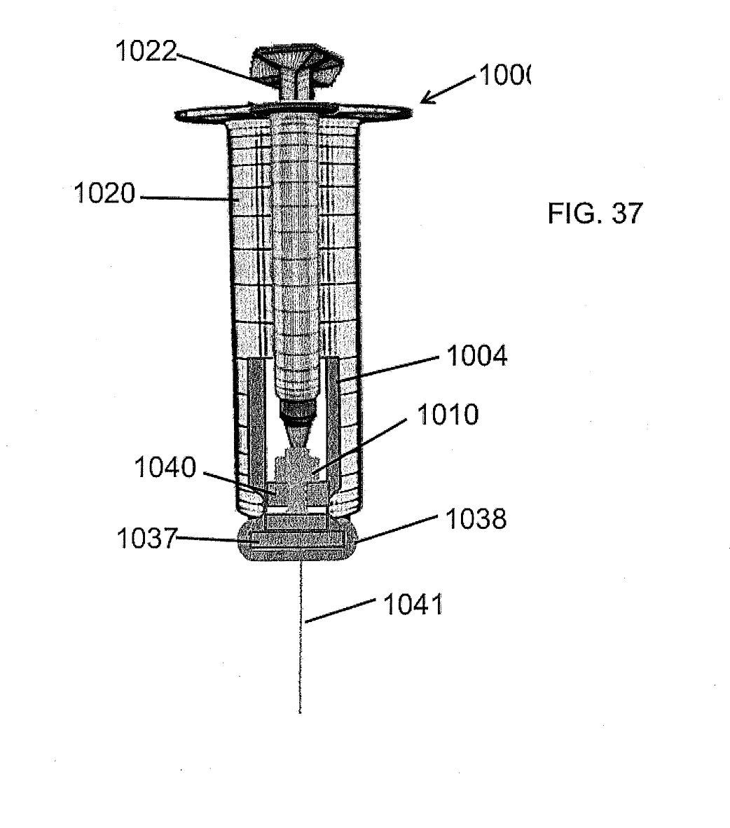

It will be understood that other orientations of reconstitution chamber (1004) may also be used such as shown in FIG. 35 where the orientation of reconstitution chamber (1004) has been reversed for use with syringe (1020) in reconstitution device (1000). Reconstitution chamber (1004) includes a proximal stopper (1040) with a luer lock (1010) having a peg (1012) inserted therein. Reconstitution chamber (1004) includes lyophilized material (1030) and a distal stopper (1037) stopper for sealing reconstitution chamber (1004). Crimped portion (1038) may be used to further seal reconstitution chamber (1004). Crimped portion (1038) is further configured to receive a needle (1041). In some versions, needle (1041) may be attached to crimped portion (1038) by simply pressing needle (1041) against crimped portion (1038), which breaches crimped portion (1038) while also sealing needle (1041) with crimped portion (1038). As seen in FIG. 35, reconstitution chamber (1004) is inserted into syringe (1020). Diluent syringe (1022) is inserted into luer lock (1010) thereby displacing peg (1012) as seen in FIG. 36. Thereafter, diluent may be injected into reconstitution chamber (1004) to reconstitute lyophilized material (1030) as seen in FIG. 36. Finally, diluent syringe (1022) may be advanced to advance proximal stopper (1040) through reconstitution device (1004) and urge fluid from needle (1041) until proximal stopper (1040) is fully advanced as seen in FIG. 37.

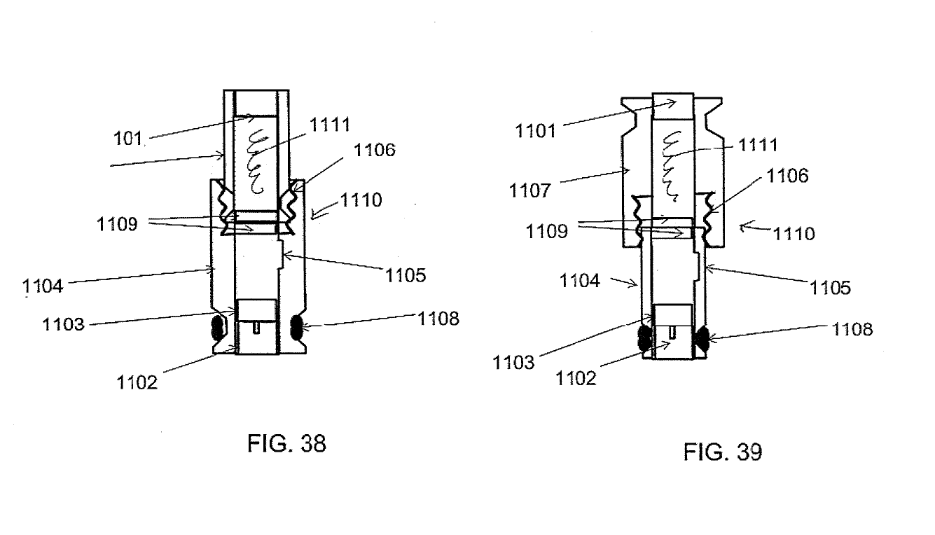

FIGS. 38-39 show an alternative variation of modes for attachment of two chambers (1104, 1107) by using an overhang of one vial to connect to another at a mated portion (1110), which could be used, for instance, as a reconstitution and diluent chamber as described above. A chamber (1104) with a lyophilized material (1103) could fit over a chamber (1107) with a liquid diluent (1111) as shown in FIG. 38 or vice versa as shown in FIG. 39. The method of attachment could involve a screw mechanism (1106), clip, plug, catch, or other means of connection. After the chambers (1104, 1107) are attached, compression of one back end stopper (1101) will cause two stoppers (1109) in the middle of chambers (1104, 1107) at the mated portion (1110) to move through the lower chamber (1104) until a means for introducing the contents of one container into a second container is reached such as a bypass loop (1105) or any other suitable structure for providing fluid communication between chambers (1104, 1107) as would be apparent to one of ordinary skill in the art. After back end stopper (1101), which may be driven by a plunger (not shown) reaches two middle stoppers (1109) located at the mated portion (1110) between two chambers (1104, 1107), force by back end stopper (1101) urges for the now mixed contents (made of diluent (1111) and lyophilized material (1103)) to be further expelled from the front of the device through a lyophilization stopper with vents (1102). It will be appreciated that either of chambers (1104, 1107) may be held within a larger container, syringe, or other structure through a sealing mechanism (1108), though sealing mechanism (1108) may in some cases be omitted. Chambers (1104, 1107) may be constructed of plastic, or any other suitable material operable to hold liquids.

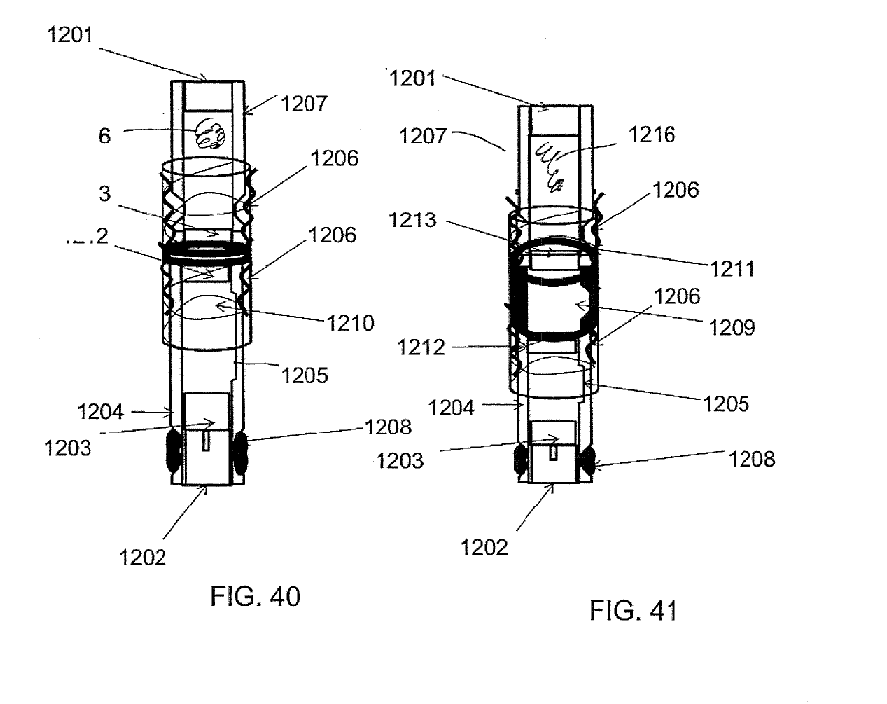

FIGS. 40-41 show yet another alternative variation of two chambers (1204, 1207) operable for use as a reconstitution chamber and diluent chamber that facilitate reconstitution and can be joined by mating component (1210, 1211). Mating component or connector (1210, 1211) can be screwed (e.g. via threads (1206)), clipped, or attached in some other means to both chambers (1204, 1207). Mating component (1210, 1211) will be such that the rubber stopper (1201) in the back of the liquid chamber (1207) traverses the liquid chamber (1207) into another solid chamber (1204) during the process of mixing and administration of the contents.

In one such modality shown in FIG. 40, the third conjoining component (1210) will only allow for attachment and movement of a back plunger (1201) and an inner plunger across it to facilitate mixing and injection of substances. In this case contents could be but should not be limited to mixing through an elongated bypass (1205) that allows material to flow around two stoppers (1212, 1213). After this flow and mixing is completed further compression allows for the delivery of mixed substances, which is made of a mixture of diluent (1216) that was held in upper chamber (1207) and lyophilized material (1203) that was held in lower chamber (1204).

In another modality shown in FIG. 41, two medicant chambers (1204, 1207) are joined by a mating component (1211) such that mating component (1211) has a built in bypass pathway (1209) of allowing liquid to flow around a lower plunger (1212) next to the conjoining section. Bypass pathway (1209) inside mating component (1211) will cause a lower plunger (1212) on the lower chamber (1207) to move as liquid flows around the lower plunger (1212) on the lower part of the upper chamber (1207). Liquid will then pass around lower plunger (1212) via a second bypass pathway (1205). The bypass pathways (1205, 1209) will be closed as the upper plunger (1213) makes its way through the full length of the upper chamber (1207) and into the lower chamber (1204) which allows for final mixing and then administration of the mixed compounds which comprises a diluent (1216) that was held in the upper chamber (1207) with a lyophilized material (1203) that was held in the lower chamber (1204). Thereafter, the combined lyophilized material (1203) and diluent (1216) may be ready for use by delivery through a syringe or any other suitable mechanism for delivery.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of any claims that may be presented and is understood not to be limited to the details of structure and operation shown and described in the specification and drawings.

I/We claim:

1. An apparatus comprising:
(a) a reconstitution chamber operable to hold a lyophilized material, wherein the reconstitution chamber is further operable to facilitate combining the lyophilized material with a diluent; and
(b) a diluent chamber operable to hold a diluent, wherein the diluent chamber is in selective fluid communication with the reconstitution chamber, wherein the reconstitution chamber and the diluent chamber are positionable within a housing.

2. The apparatus of claim 1, wherein a wall portion of the reconstitution chamber defines a cutout operable to act as a bypass for the diluent to enter the reconstitution chamber from the diluent chamber.

3. The apparatus of claim 1, wherein the diluent chamber includes at least one moveable stopper, wherein the at least one moveable stopper is operable to move axially through the diluent chamber into at least a portion of the reconstitution chamber.

4. The apparatus of claim 1, wherein the reconstitution chamber is configured to be movable within the housing, wherein the reconstitution chamber is further configured to form a fluid seal with the housing as the reconstitution chamber moves through the housing.

5. The apparatus of claim 1, further comprising a stopper configured to provide a fluid seal between the reconstitution chamber and the diluent chamber, wherein an external force applied to the diluent chamber is operable to dislodge the stopper in a direction opposing the direction with which the external force is applied.

6. The apparatus of claim 1, further comprising a rod extending through the reconstitution chamber, wherein the rod is movable between a first position and a second position, wherein the rod in the first position is configured to obstruct fluid flow between the reconstitution chamber and the diluent chamber, wherein the rod in the second position is configured to establish fluid communication between the reconstitution chamber and the diluent chamber.

7. The apparatus of claim 6, wherein a plunger is configured to advance the reconstitution chamber within the housing once the rod is in the second position.

8. The apparatus of claim 1, further comprising a thread connected with a seal, wherein the thread is configured to be routed out of a needle, wherein the seal is positioned between the reconstitution chamber and the diluent chamber.

9. The apparatus of claim 1, further comprising a thread and a stopper wherein the thread is connected to a plunger of a syringe, wherein the thread is also connected to the stopper, wherein the plunger is configured to be actuated to break the connection of the thread between the stopper and the plunger.

10. The apparatus of claim 1, wherein the reconstitution chamber is positioned in a syringe, wherein a diluent syringe is in selective fluid communication with the reconstitution chamber, wherein the diluent syringe includes a diluent plunger moving telescopically within the diluent syringe, wherein the diluent syringe is operable to move telescopically within the syringe.

11. The apparatus of claim 1, further comprising a plunger having a pointed tip and a breakable seal positioned between the reconstitution chamber and the diluent chamber, wherein the plunger is configured to be advanced to pierce the breakable seal.

12. The apparatus of claim 11, wherein the pointed tip has a cone shape.

13. The apparatus of claim 11, wherein the pointed tip includes an off center post.

14. The apparatus of claim 1, further comprising two bypass regions staggered along the length of the reconstitution chamber configured to provide fluid communication between the reconstitution chamber and the diluent chamber.

15. The apparatus of claim 1, further comprising a seal and a proximally facing piercing tip, wherein the seal is positioned between the reconstitution chamber and the diluent chamber, wherein the seal is configured to advance against the piercing tip.

16. An apparatus comprising:
(a) a syringe, wherein at least a portion of the syringe is operable to hold a diluent; and
(b) a reconstitution chamber contained within the syringe, wherein the reconstitution chamber includes a medicament for reconstitution, wherein the reconstitution chamber is configured to selectively provide fluid communication with the portion of the syringe operable to hold a diluent.

17. The apparatus of claim 16, further comprising a stopper positioned between the reconstitution chamber and the portion of the syringe operable to hold the diluent, wherein the stopper is configured to dislodge by rotating the syringe which is screwingly in communication with the stopper.

18. The apparatus of claim 16, further comprising a stopper and a rod, wherein the stopper is positioned between the reconstitution chamber and the portion of the syringe operable to hold the diluent, wherein the rod is configured to selectively plug the stopper.

19. A reconstitution chamber comprising:
(a) a chamber cartridge, wherein the chamber cartridge is configured to hold a drug, wherein the chamber cartridge has a generally cylindrical shape, wherein the chamber cartridge defines a first opening and a second opening, wherein the first opening and the second opening are opposingly positioned along the chamber cartridge;
(b) a first stopper positioned within the first opening; and
(c) a second stopper positioned within the second opening, wherein the chamber cartridge is positionable within a housing, wherein the drug is operable to be urged out of the chamber cartridge through one of the first opening or the second opening.

20. The reconstitution chamber of claim 19, wherein the chamber cartridge includes uniform internal walls along the length of the chamber cartridge, wherein the chamber cartridge is operable to receive a plunger operable to urge the drug from the chamber cartridge by moving through at least a portion of the length of the chamber cartridge.
